(19) United States
(12) Patent Application Publication
(10) Pub. No.: US 2017/0164913 A1
(43) Pub. Date: Jun. 15, 2017

(54) RADIOGRAPHY SYSTEM

(71) Applicant: Konica Minolta, Inc., Tokyo (JP)

(72) Inventors: SHIN SHINOTSUKA, Tokyo (JP); EMIEL VISSER, Tokyo (JP)

(21) Appl. No.: 15/378,645

(22) Filed: Dec. 14, 2016

(30) Foreign Application Priority Data
Dec. 15, 2015 (JP) 2015-244116

(51) Int. Cl. A61B 6/08; A61B 6/00; A61B 6/06

(52) U.S. Cl. CPC A61B 6/08; A61B 6/06; A61B 6/4283; A61B 6/4405

(57) ABSTRACT

A radiation generator capable of projecting radiation includes a light projection apparatus configured to project visible light that indicates, during projection of radiation from the radiation generator, a range including and broader than an irradiation field of radiation projected from the radiation generator.

### CROSS REFERENCE TO RELATED APPLICATIONS

The entire disclosure of Japanese Patent Application No. 2015-244116 filed on Dec. 15, 2015 including description, claims, drawings, and abstract are incorporated herein by reference in its entirety.

### BACKGROUND OF THE INVENTION

Field of the Invention

The present invention relates to a radiography system, particularly relates to a radiography system including a radiation generator mounted on a round-visit wagon and including a portable radiation generator.

Description of the Related Art

As a radiography apparatus to replace conventional films/screens, stimulable phosphor plates, or the like, a flat panel detector (FPD) cassette has been developed. As illustrated in FIG. 2 described below, for example, the flat panel detector (FPD) cassette includes a plurality of radiation detecting elements 7 arranged two-dimensionally (in a matrix) and configured to convert, on each of the radiation detecting elements 7, radiation transmitted through a subject, into image data D in accordance with its intensity and configured to read the data.

When radiation is projected a plurality of times to the conventional film/plate or the stimulable phosphor plate, problems such as double exposure or multiple exposure would arise, hindering performing moving image photographing of the subject. In contrast, the FPD cassette is capable of storing the image data D that have been read, into a storage unit 23 inside the apparatus (refer to FIG. 2 to be described below), and thus, enables performing moving image photographing such as kinetic photographing, of the subject.

The kinetic photographing is photographing in which radiation is projected to the chest of the patient as a subject, for example, a plurality of times, and individual radiographic images (also referred to as a frame image, etc. in the case of kinetic photographing) of individual time phases T (T=t₀ to t₆) of a lung field R of the patient are obtained, as illustrated in FIG. 9, for example. In addition, there have been attempts to perform further analyzation, or the like, of those radiographic images so as to apply the images to diagnosis. In the present invention, moving image photographing is not limited to the above-described kinetic photographing but may include any mode as long as it is photographing in which radiation is projected to the subject a plurality of times, or projected to the subject consecutively, and a plurality of radiographic images are photographed.

Moving image photographing using these FPD cassettes may be performed in a well-equipped photographing room in some cases. In another case where the patient has difficulty in coming to the photographing room, however, a round-visit wagon (refer to JP 2003-310595 A, JP 2001-57972 A, JP 2014-204783 A, etc.) equipped with an FPD cassette, a radiation generator, or the like, would be brought into a patient room of a hospital or clinic. In this case, moving image photographing would be performed by projecting radiation a plurality of times onto a photographing site such as the chest of the patient as a subject.

Furthermore, there may be a case where a portable radiation generator 90 as illustrated in FIG. 10 is brought into the patient room and moving image photographing is performed. Note that while FIG. 10 illustrates merely a main body portion of the portable radiation generator 90, the portable radiation generator 90 is fixed onto a base or legs (not illustrated), or the like, in practical use.

Meanwhile, in a case where moving image photographing such as kinetic photographing is performed, a photographer such as a radiologist needs to cautious about, by continuous monitoring, whether the posture of the patient as the subject is appropriately maintained during photographing. Unlike a case of performing kinetic photographing in a photographing room, in a case where photographing is performed in a situation where a radiation generator mounted on a round-visit wagon, a portable radiation generator, or the like, is brought into the patient room, there is a possibility that a person, or the like, other than the patient as the subject (hereinafter, simply referred to as a nearby person) might enter an irradiation field of radiation and be exposed.

To cope with this, the photographer needs to be further cautious about, for example, preventing the nearby person from entering the irradiation field of radiation. In practice, however, it would not necessarily be easy to be cautious about preventing the nearby person from entering the irradiation field of radiation while monitoring the patient as the subject.

On the other hand, however, unlike plain radiography in which projection of radiation is finished instantaneously, radiation is continuously projected from the radiation generator onto the subject for about 20 seconds, for example, in the case of performing moving image photographing such as kinetic photographing. While projection of radiation is configured to be notified, in many cases, by lighting of an indication lamp or sound during projection of radiation from the radiation generator, there may be not a few cases where the nearby person has difficulty in discerning whether the indication lamp is turned on, or in catching the sound, and thus, has difficulty in discerning whether the radiation is projected or finished.

In addition, since radiation is invisible, there are many cases in which the nearby person does not know in what range the radiation is projected. Because of these, there may be problems such as the nearby person feels too nervous, for example, has a false recognition of being prohibited from a range that is actually a permitted range, and thus, feels own action is restricted than necessary so as to avoid, for example, entering the area. In another case, there may be a problem that the nearby person believes that the projection of radiation is finished and enters the irradiation field of radiation and is exposed.

In this manner, particularly when a radiation generator mounted on a round-visit wagon, a portable radiation generator, or the like, is brought into the patient room and moving image photographing such as kinetic photographing is performed with this radiation generator, it is necessary to be cautious about the difference of photographing environment from the cases of photographing in the photographing room in a hospital, or the like, in that there is a possibility that not merely the patient and a photographer such as a radiologist but also a nearby person, that is, a person other than the patient as a subject, is present in the vicinity of a photographing site.

### SUMMARY OF THE INVENTION

The present invention has been made in view of this issue, and an object thereof is to provide a radiography system capable of allowing a patient as a subject and a person other than the patient to properly recognize the projection range of radiation and whether the projection of radiation is finished, in performing moving image photographing by bringing a radiation generator mounted on a round-visit wagon or a portable radiation generator, into the patient room.

To achieve the abovementioned object, according to an aspect, a radiation generator capable of projecting radiation, reflecting one aspect of the present invention comprises
a light projection apparatus configured to project visible light that indicates, during projection of radiation from the radiation generator, a range including and broader than an irradiation field of radiation projected from the radiation generator.

To achieve the abovementioned object, according to an aspect, a round-visit wagon for radiography reflecting one aspect of the present invention comprises
a radiation generator capable of projecting radiation, wherein
the radiation generator includes a light projection apparatus configured to project visible light that indicates, during projection of radiation from the radiation generator, a range including and broader than an irradiation field of the radiation projected from the radiation generator.

To achieve the abovementioned object, according to an aspect, a radiography system reflecting one aspect of the present invention comprises:

an FPD cassette including a plurality of radiation detecting elements arranged two-dimensionally; and

a radiation generator capable of projecting radiation, wherein

the radiation generator includes a light projection apparatus configured to project visible light that indicates, during projection of radiation from the radiation generator, a range including and broader than an irradiation field of the radiation projected from the radiation generator.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a perspective view illustrating appearance of an FPD cassette;

FIG. 2 is a block diagram illustrating an equivalent circuit of the FPD cassette;

FIG. 3 is a perspective view illustrating appearance of a round-visit wagon according to the present embodiment;

FIG. 4 is a block diagram illustrating a configuration of a console;

FIG. 5A is a diagram illustrating a general configuration of a radiography system according to the present embodiment;

FIG. 5B is a diagram illustrating an irradiation field of radiation and an irradiation range of visible light, that is broader than the irradiation field of radiation;

FIG. 6 is a diagram illustrating a configuration of an irradiation field lamp, or the like, within a collimator unit;

FIG. 7 is a diagram illustrating am exemplary configuration of a light projection apparatus;

FIG. 8A is a diagram illustrating a state in which a detection apparatus is attached to an exposure switch;

FIG. 8B is a diagram illustrating a state in which a button on the exposure switch is pressed;

FIG. 9 is a diagram illustrating an exemplary frame image photographed in kinetic photographing of the chest of a patient; and

FIG. 10 is a diagram illustrating an exemplary portable radiation generator.

### DESCRIPTION OF THE PREFERRED EMBODIMENTS

Hereinafter, a radiography system according to an embodiment of the present invention will be described with reference to the drawings. However, the scope of the invention is not limited to the illustrated examples.

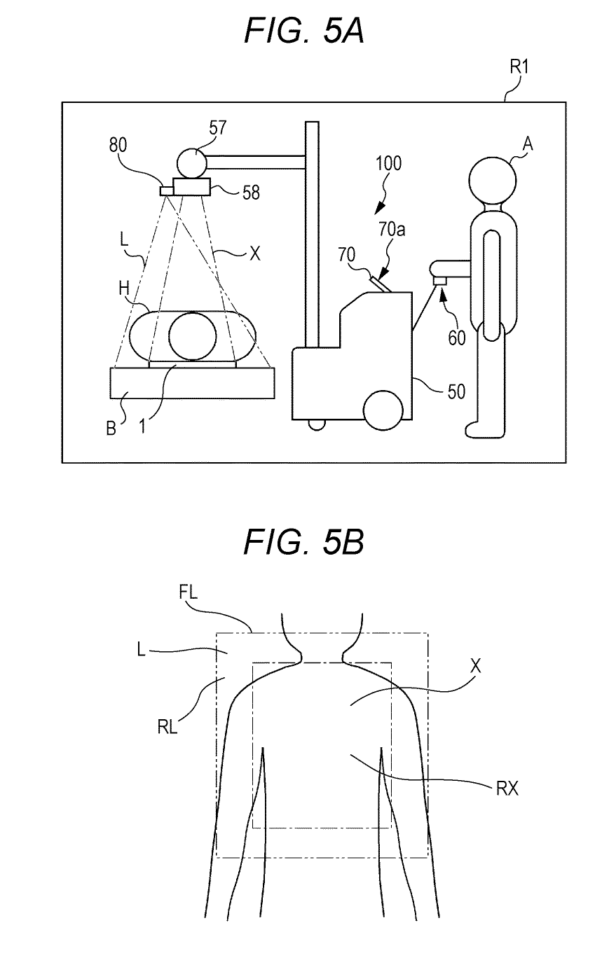

In the following description, moving image photographing such as kinetic photographing is performed in a state in which, a patient as a subject H is lying on a bed B, or the like, as illustrated in FIG. 5A described below, for example. The present invention, however, is also applicable to moving image photographing performed in a state in which the patient is in a seated position on the bed B or standing (in a standing position).

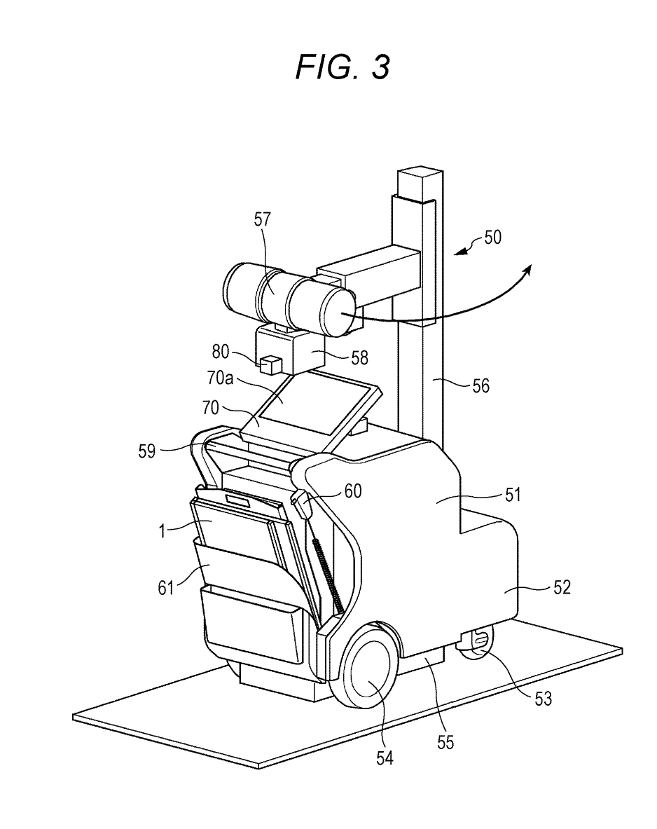

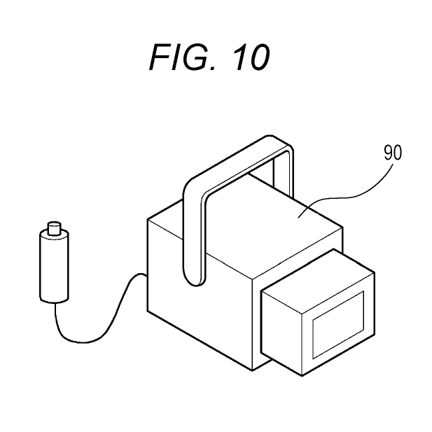

In the following description, moving image photographing is performed in a state in which a radiation generator 57 mounted on a round-visit wagon 50 (refer to FIGS. 3 and 5A described below) is brought into the patient room. The present invention, however, is applicable to a case where, as described above, moving image photographing is performed in a state where the portable radiation generator 90 (refer to FIG. 10) is brought into the patient room, and this case is also included in the present invention.

[Configuration of FPD Cassette]

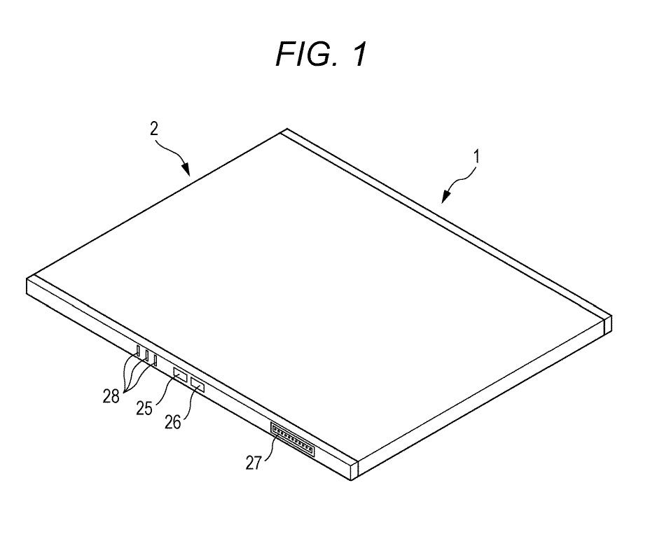

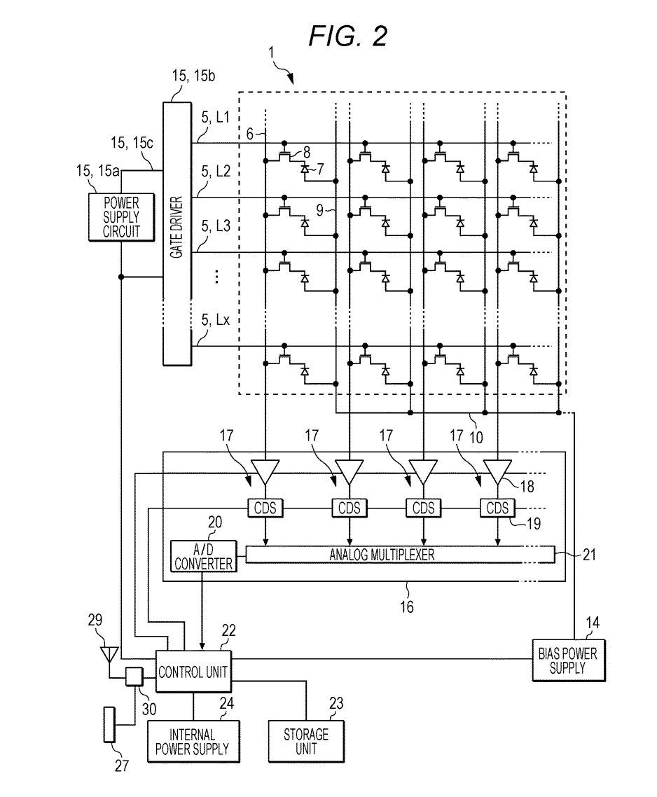

First, a configuration of an FPD cassette will be briefly described. FIG. 1 is a perspective view illustrating appearance of the FPD cassette. FIG. 2 is a block diagram illustrating an equivalent circuit of the FPD cassette. As illustrated in FIGS. 1 and 2, an FPD cassette 1 is configured such that a plurality of radiation detecting elements 7 two-dimensionally arranged (in a matrix) on a sensor substrate (not illustrated) is contained in a casing 2.

Note that FIG. 1 illustrates an exemplary case in which a power switch 25, changeover switch 26, a connector 27, an indicator 28, or the like, are arranged on one side surface of the casing 2 of the FPD cassette 1. Although not illustrated, an antenna 29 (refer to FIG. 2 described below) for wirelessly communicating with the outside is provided, for example, on a side surface on an opposite side of the casing 2.

As illustrated in FIG. 2, each of the radiation detecting elements 7 is connected with a bias line 9, and reverse bias voltage is applied from a bias power supply 14 to the radiation detecting element 7 via the bias line 9 and connection 10 thereof. Each of the radiation detecting elements 7 is connected with a thin film transistor (TFT) 8 as a switching element. The TFT 8 is connected with a signal line 6.

In a scan drive unit 15, on-voltage and off-voltage supplied from a power supply circuit 15a via wiring 15c are switched over at a gate driver 15b, so as to be applied to each of lines L1 to Lx of a scan line 5. When the off-voltage is applied to each of the TFTs 8 via the scan line 5, each of the TFTs 8 turns off, interrupting the conduction between the radiation detecting element 7 and the signal line 6, so as to allow electrical charges to be stored in the radiation detecting elements 7. When the on-voltage is applied to each of the TFTs 8 via the scan line 5, each of the TFTs 8 is turned on, so as to allow the electrical charges stored in the radiation detecting elements 7 to be discharged to the signal line 6.

Each of the signal lines 6 is connected to each of readout circuits 17 inside a readout IC 16. At the time of readout processing of image data D, on-voltage is sequentially applied from the gate driver 15b to each of the lines L1 to Lx of the scan line 5. When the TFT 8 is turned on, electrical charges flow from the radiation detecting elements 7 into the readout circuit 17 via the TFT 8 and the signal line 6. A voltage value corresponding to the amount of electrical charges that flow into the circuit is output at an amplification circuit 18.

A correlated double sampling circuit (described as “CDS” in FIG. 2) 19 reads a voltage value output from the amplification circuit 18 as the image data D in analog values, and outputs the data. The output image data D are sequentially transmitted to an A/D converter 20 via an analog multiplexer 21, sequentially converted at the A/D converter 20, into the image data D in digital values, and sequentially stored in the storage unit 23.

A control unit 22 includes a computer in which, a central processing unit (CPU), a read only memory (ROM), a random access memory (RAM), an input/output interface, or the like (none of them being illustrated), are connected to a bus, a field programmable gate array (FPGA). The control unit 22 may include a dedicated control circuit.

The control unit 22 is connected with a storage unit 23 and an internal power supply 24. The storage unit 23 includes a static RAM (SRAM), a synchronous DRAM (SDRAM), and a NAND-type flash memory. The internal power supply 24 includes a lithium ion capacitor. The control unit 22 is also connected with a communication unit 30 for performing wireless or wired communication with the outside via the above-described antenna 29 and the connector 27.

Moreover, as described above, the control unit 22 controls application of reverse bias voltage from the bias power supply 14, to each of the radiation detecting elements 7. The control unit 22 also controls operation of the scan drive unit 15, the readout circuit 17, or the like, to perform readout processing toward the image data D from each of the radiation detecting elements 7. The control unit 22 also performs controls such as storing the image data D in the storage unit 23, or transfer of the stored image data D to the outside via the communication unit 30.

[Round-Visit Wagon]

Next, a round-visit wagon according to the present embodiment will be described. FIG. 3 is a perspective view illustrating appearance of the round-visit wagon according to the present embodiment. In the following description, the side on which a support column 56 of the radiation generator 57 is installed is referred to as a front side of the round-visit wagon 50, the side on which a handle bar 61 is installed is referred to as a rear side of the round-visit wagon 50.

In the present embodiment, as illustrated in FIG. 3, the round-visit wagon 50 includes a base 52 covered with a cover 51, with two front wheels 53 being provided at a front portion of the base 52, two rear wheels 54 being provided at a rear portion of the base 52. Drivers such as a motor (not illustrated) provided for rotatably driving the rear wheels 54 are incorporated in the main body 55 covered with the cover 51. Moreover, a control section (not illustrated), or the like, of the radiation generator 57 described below is also incorporated in the main body 55.

The support column 56 of the radiation generator 57 is provided in an erected state, in substantially a vertical direction, on a front side of the base 52 of the round-visit wagon 50. In the present embodiment, the support column 56 is expandable in an up-down direction. With expansion of the support column 56 in the up-down direction, the radiation generator 57 mounted on an upper end of the support column 56 also moves upwardly and downwardly. Alternatively, it is also possible to configure such that the support column 56 does not move in the up-down direction but the radiation generator 57 is lifted up and down along the support column 56.

In the present embodiment, with pivoting movement of the support column 56 around its extending direction (namely, up-down direction), the radiation generator 57 can also pivot around the extending direction of the support column 56 (refer to the arrow in FIG. 3) . Alternatively, it is also possible to configure such that the radiation generator 57 is pivoted as described above by fixing the radiation generator 57 to the support column 56 and by pivoting the support column 56.

In conveyance of the round-visit wagon 50, the round-visit wagon 50 is conveyed in a state where the radiation generator 57 is pivoted to a portion above the main body 55 (state illustrated in FIG. 3) . When radiation is projected from the radiation generator 57, the round-visit wagon 50 is configured to be used in a state where the radiation generator 57 is pivoted by a predetermined angle, for example, 180° from the state illustrated in FIG. 3.

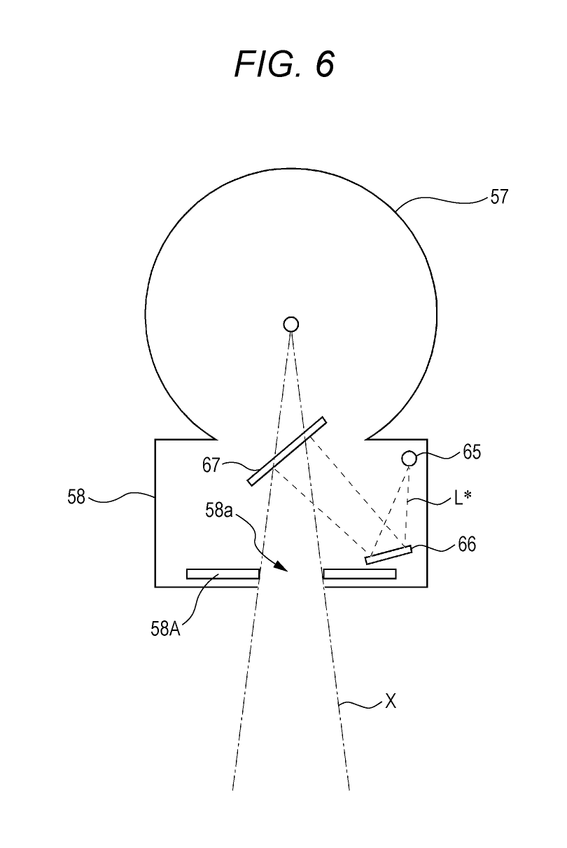

It is possible use a known device including a rotating anode (not illustrated), as the radiation generator 57. A collimator unit 58, or the like, is mounted below the radiation generator 57. The collimator unit 58 incorporates a collimator 58A (refer to FIG. 6 described below), or the like. The collimator 58A is configured to narrow an irradiation field for the radiation projected from the radiation generator 57. A light projection apparatus 80 mounted on the collimator unit 58 of the radiation generator 57 will be described below. Alternatively, the light projection apparatus 80 may be mounted on a main body or the support column 56, of the radiation generator 57.

A handle bar 59 is provided at an upper portion behind the round-visit wagon 50. The handle bar 59 is gripped by a photographer such as a radiologist when he or she moves the round-visit wagon 50, for example. In addition, an exposure switch 60 is removably attached at a side portion behind the round-visit wagon 50. The exposure switch 60 is configured to instruct projection of radiation from the radiation generator 57, to a control section of the radiation generator 57 within the main body 55.

In the present embodiment, a cassette holding unit 61 is provided at a rear-end portion of the round-visit wagon 50. The FPD cassette 1, or the like, is insertable into the cassette holding unit 61. At the conveyance of the round-visit wagon 50 to a patient room, it is conveyed in a state where the FPD cassette 1, or the like, is inserted into the cassette holding unit 61, thereby making it possible to reduce time and effort for the photographer such as a radiologist to carry the FPD cassette 1.

Meanwhile, a console 70 is arranged at an upper surface portion of the cover 51 of the round-visit wagon 50. The console 70 is configured to perform processing such as control of operation of the FPD cassette 1, image processing of the image data D transferred from the FPD cassette 1, as described above, to generate a radiographic image, and displaying the generated radiographic image on a display screen 70a.

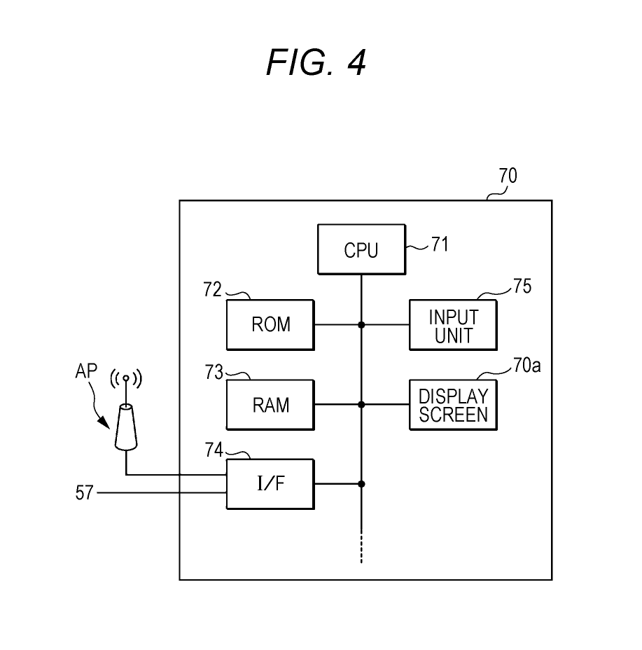

In the present embodiment, the console 70 is configured as a computer in which, as illustrated in FIG. 4, a CPU 71, a ROM 72, a RAM 73, an input/output interface 74, or the like, are connected to a bus. The console 70 is also connected to an input unit 75 such as a keyboard, a mouse, and a touch panel, and the above-described display screen 70a including a cathode ray tube (CRT), a liquid crystal display (LCD), or the like. The console 70 is further connected with an access point AP, or the like, for performing wireless communication with the FPD cassette 1, or the like, via the input/output interface 74.

In the present embodiment, the console 70 is also connected with the control section of the radiation generator 57 via the input/output interface 74, and setting of tube voltage, tube current, photographing time (or mAs value) , or the like, toward the control section of the radiation generator 57 can be performed on the console 70. The configuration, however, need not necessarily be like this. As will be similarly applied to the description below, the photographing time in this case represents the entire time from the start of projection till the end of projection, of the radiation projected in pulses or radiation projected continuously from the radiation generator 57. In a case where the radiation is projected a plurality of times in pulses, the projection time for one pulse (that is, time from the start of projection till the end of projection, for one-pulsed radiation) is separately set.

While FIG. 3 illustrates an exemplary case in which the console 70 is configured integrally with the round-visit wagon 50, it is also possible to configure such that the console 70 including a notebook computer is mounted on the round-visit wagon 50. Moreover, as described below, in a case where the portable radiation generator 90 (refer to FIG. 10), or the like, is brought into a patient room R1 for performing photographing, also the console 70 including the notebook computer, the console 70 including a mobile terminal, or the like, is brought into the patient room R1.

[Configuration Unique to the Present Invention]

Next, a configuration, or the like, unique to the present invention in a radiography system 100 according to the present embodiment will be described. In addition, actions of the radiography system 100 according to the present embodiment will also be described.

In the radiography system 100 according to the present embodiment, moving image photographing, or the like, is performed by bringing the round-visit wagon 50 into the patient room R1, for example, as illustrated in FIG. 5A. Note that illustrations of other bed B in the patient room. R1, and a person, or the like, other than the patient as the subject H and other than the photographer A such as a radiologist, are omitted in FIG. 5A. The description will be applied similarly to a case where photographing is performed by bringing the portable radiation generator 90 (refer to FIG. 10), instead of a case where photographing is performed by bringing the radiation generator 57 mounted on the round-visit wagon 50.

First, a procedure of ordinary moving image photographing, or the like, will be described. The photographer A such as a radiologist prepares for photographing, specifically, sets the FPD cassette 1 by inserting the FPD cassette 1 between a photographing site (for example, chest) of the patient as the subject H and the bed B, for example. Subsequently, the radiation generator 57 of the round-visit wagon 50 is moved upward/downward, or pivotally, and the round-visit wagon 50 is moved close to the bed B such that the radiation generator 57 is arranged above the subject H. Subsequently, positioning operation is performed including position adjustment of the FPD cassette 1, positioning of the radiation generator 57, or adjustment of the narrowing level of the collimator, as necessary.

When the positioning is finished, the photographer A moves to the rear side of the round-visit wagon 50 and performs moving image photographing by operating the exposure switch 60 to project radiation from the radiation generator 57. The radiation projected from the radiation generator 57 is incident onto the FPD cassette 1 through the subject H, a one-frame portion of the image data D is photographed with the FPD cassette 1. This photographing is performed a plurality of times and thus, moving image photographing such as kinetic photographing is performed by photographing a plurality of frames of image data D.

At this time, it is possible to configure such that the image data Dare transferred from the FPD cassette 1 to the console 70 for each of photographing (that is, each of the frames), or it is also possible to configure such that a plurality of frames of image data D is transferred, at one time, to the console 70 at a point where a series of plurality of times of photographing is finished.

In the present embodiment, radiation is continuously projected from the radiation generator 57 during the time for which the photographer A continuously presses the exposure switch 60. After a predetermined period of photographing has elapsed, projection of radiation from the radiation generator 57 is automatically finished even when the photographer A continuously presses the exposure switch 60. Moreover, it is configured such that projection of radiation from the radiation generator 57 can be stopped when the photographer A stops pressing the exposure switch 60, even though the predetermined period of time has not elapsed after starting projection of radiation.

[Indicating, by Visible Light, Range Including and Broader than Irradiation Field of Radiation]

In the radiography system according to the present embodiment, the light projection apparatus 80 capable of projecting visible light is attached on the radiation generator 57 or the collimator unit 58 thereof, as described above, on the round-visit wagon 50.

In the present embodiment, as illustrated in FIGS. 5A and 5B, it is configured to project visible light L from the light projection apparatus 80 during projection of radiation X from the radiation generator 57 to the subject H, thereby indicating, by the visible light L, a range RL including and broader than an irradiation field RX (refer to FIG. 5B) of the radiation X projected from the radiation generator 57 to the subject H.

As described above, the person other than the patient as the subject H (nearby person) has difficulty in discerning turn-on of the indication lamp and generation of sound, or the like, that indicate the state of projection of the radiation X. Because of this, the nearby person has difficulty in discerning whether the radiation X is projected, and since radiation X is invisible, it is not easy to discern in what region (irradiation field RX) the radiation X is projected, even when the radiation X is projected from the radiation generator 57 to the subject H.

To overcome this issue, as illustrated in the present embodiment, it would be configured such that the visible light L is projected from the light projection apparatus 80 during projection of the radiation X from the radiation generator 57 to the subject H, and thus, it would be configured to indicate, by the visible light L, the range RL including and broader than the irradiation field RX of the radiation X projected from the radiation generator 57 to the subject H. With this configuration, the nearby person can recognize that the radiation X is projected within the range RL (actually, within the irradiation field RX of the radiation X that is smaller than the range RL) indicated by the visible light L projected from the light projection apparatus 80.

Furthermore, with the configuration according to the present embodiment, the nearby person can recognize a fact that the radiation X is projected by confirming that the visible light L is projected, and can recognize that the radiation X is not projected by confirming that the visible light L is not projected, and thus, can properly recognize whether the projection of radiation is finished.

[Configuration of Light Projection Apparatus—Part 1]

Meanwhile, in the present embodiment, an irradiation field lamp 65 capable of projecting light L* is provided within the collimator unit 58 of the radiation generator 57, as illustrated in FIG. 6. The irradiation field lamp 65 is configured to project, before photographing, light to the subject H, specifically, to a region to be the irradiation field RX of the radiation X when the radiation X is projected (that is, a range same as the irradiation field RX of the radiation X).

More specifically, the radiation generator 57 includes the irradiation field lamp 65, and mirrors 66 and 67, within the collimator unit 58. Before photographing (that is, before performing photographing by projecting the radiation X from the radiation generator 57), a mirror 67 is arranged on an optical axis of the radiation X to be projected from the radiation generator 57 at photographing to be performed thereafter, and then, the irradiation field lamp 65 is lit, and the light L* projected from the irradiation field lamp 65 is reflected in a projection direction of the radiation X, at the mirrors 66 and 67.

The reflected light L* is narrowed in range at the collimator 58A, and thereafter, is projected to the subject H (not illustrated in FIG. 6). At this time, the projection range of the light L* toward the subject H indicates the irradiation field RX of the radiation X to be projected at photographing to be performed thereafter.

Accordingly, the photographer A such as a radiologist turns on the irradiation field lamp 65 before photographing, and preparation operation such as positioning of the radiation generator 57 with respect to the subject H, narrowing adjustment of the collimator 58A, or the like, is performed while the light L* projected to the subject H is monitored. After the positioning is finished, photographing is performed by projecting the radiation X from the radiation generator 57.

In this manner, the light L* is projected to the irradiation field RX of the radiation X from the irradiation field lamp 65 before photographing. By using this procedure, it would be configured such that the light L* is projected from the irradiation field lamp 65 also in a period in which the radiation X is projected from the radiation generator 57 for photographing. With this configuration, it is possible to indicate the irradiation field RX of the radiation X projected onto the subject H from the radiation generator 57 by irradiating the field with the light L* projected from the irradiation field lamp 65.

On the other hand, however, there is a concern that, since the radiation X projected from the radiation generator 57 and the light L* projected from the irradiation field lamp 65 are both narrowed by the identical collimator 58A during photographing, the irradiation field RX of the radiation X and the range of the light L* projected from the irradiation field lamp 65 would be a same range.

Therefore, in a case where, as described above, the light L* projected from the irradiation field lamp 65 is configured to be narrowed by the same collimator as the collimator 58A configured to narrow the radiation X, it would not be possible to indicate the range RL (refer to FIG. 5B) including and broader than the irradiation field RX of the radiation X projected from the radiation generator 57 to the subject H, during photographing, by the light L* as described in an embodiment of the present invention. In other words, with the above-described configuration, it would be difficult to use the irradiation field lamp 65 as the light projection apparatus 80 according to an embodiment of the present invention.

Accordingly, it would be difficult to use the irradiation field lamp 65 with the above-described configuration (refer to FIG. 6) as the light projection apparatus 80 according to an embodiment of the present invention, and thus, the light projection apparatus 80 according to an embodiment of the present invention is to be configured as an apparatus separate from the irradiation field lamp 65.

Incidentally, however, there are cases where the irradiation field lamp 65 is not provided within the collimator unit 58 (unlike the case of FIG. 6) and the irradiation field lamp 65 is attached to a portion outside of the collimator unit 58, to the radiation generator 57, or to the support column 56 of the radiation generator 57, as illustrated with the light projection apparatus 80 in FIGS. 3 and 5A, for example.

With this configuration, the light L* projected from the irradiation field lamp 65 is not narrowed by the collimator 58A configured to narrow the radiation X (that is, the light L* is not transmitted through the collimator 58A). Accordingly, it is possible to project the light L* from the irradiation field lamp 65 and to indicate, by the light L*, the range RL (refer to FIG. 5B) including and broader than the irradiation field RX of the radiation X projected from the radiation generator 57 to the subject H.

Therefore, in a case where, as described above, the irradiation field lamp 65 is attached to a portion outside of the collimator unit 58, to the radiation generator 57, to the support column 56 of the radiation generator 57, or the like, it would be possible to use the irradiation field lamp 65 as the light projection apparatus 80 according to an embodiment of the present invention.

Moreover, even when the irradiation field lamp 65 is provided within the collimator unit 58, in a case where it is configured such that the light L* projected from the irradiation field lamp 65 is not narrowed by the collimator 58A configured to narrow the radiation X (that is, the light L* is not transmitted through an aperture 58a of the collimator 58A (refer to FIG. 6), it would be possible to project the light L* from the irradiation field lamp 65 and to indicate, by the light L*, the range RL (refer to FIG. 5B) including and broader than the irradiation field RX of the radiation X projected from the radiation generator 57 to the subject H. Accordingly, it is possible to use the irradiation field lamp 65 as the light projection apparatus 80 according to an embodiment of the present invention.

In this case, it is configured such that, positioning, or the like, of the radiation generator 57 with respect to the subject H is performed before photographing on the basis of the light L* projected from the irradiation field lamp 65, and thereafter, the range RL (refer to FIG. 5B) including and broader than the irradiation field RX of the radiation X is indicated by the light L* by expanding, during photographing, the projection range of the light L*(corresponding to the visible light L) projected from the irradiation field lamp 65 as the light projection apparatus 80 as necessary, for example.

In this manner, not merely in an understandable case where the irradiation field lamp 65 is attached on a portion outside of the collimator unit 58, that is, on the outside of the collimator unit 58 itself, on the radiation generator 57, or on the support column 56 of the radiation generator 57, similarly to the light projection apparatus 80 as illustrated in FIGS. 3 and 5A, but also in a case where, while the irradiation field lamp 65 is provided within the collimator unit 58, the irradiation field lamp 65 is provided at a position where the light L* projected from the irradiation field lamp 65 is not transmitted through the aperture 58a of the collimator 58A, it would also be possible to use the irradiation field lamp 65 as the light projection apparatus 80 according to the present embodiment.

Even in a case where the light projection apparatus 80 is arranged as an apparatus separate from the irradiation field lamp 65, it would also be possible to indicate the range RL (refer to FIG. 5B) including and broader than the irradiation field RX of the radiation X projected from the radiation generator 57, by the visible light L projected from the light projection apparatus 80, provided that the light projection apparatus 80 is arranged on the outside of the collimator unit 58 (that is, outside of the collimator unit 58 itself, on the radiation generator 57, or on the support column 56 of the radiation generator 57), or at a position where the visible light L projected from the light projection apparatus 80 is not transmitted through the aperture 58a of the collimator 58A in a case where the light projection apparatus 80 is arranged within the collimator unit 58.

In other words, the light projection apparatus 80 according to the present embodiment (including a case of using the irradiation field lamp 65) is provided at a position where the visible light L projected from the light projection apparatus 80 is not transmitted through the aperture 58a of the collimator 58A configured to narrow the irradiation field RX of the radiation X projected from the radiation generator 57, in any of the cases where the light projection apparatus 80 is provided outside of the collimator unit 58 and within the collimator unit 58.

With this configuration, the visible light L projected from the light projection apparatus 80 is not narrowed by the collimator 58A configured to narrow the irradiation field RX of the radiation X. Accordingly, it is possible to indicate the range RL including and broader than the irradiation field RX of the radiation X projected from the radiation generator 57, by the visible light L projected from the light projection apparatus 80.

Note that embodiments of the present invention are not intended to exclude performing, before photographing, the above-described operation such as positioning of the radiation generator 57 with respect to the subject H, or the narrowing level adjustment of the collimator 58A, or the like, by projecting the light L* from the irradiation field lamp 65 to the subject H. In other words, in case of using the irradiation field lamp 65 of the type according to the present embodiment illustrated in FIG. 6, preparation operation such as positioning of the radiation generator 57 with respect to the subject H, narrowing level adjustment of the collimator 58A, is performed before photographing by projecting the light L* from the irradiation field lamp 65.

After finishing the preparation operation, the photographer A such as a radiologist turns off the irradiation field lamp 65 and performs photographing in a state where the visible light L is projected from the light projection apparatus 80 to the subject H.

[Configuration of Light Projection Apparatus—Part 2]

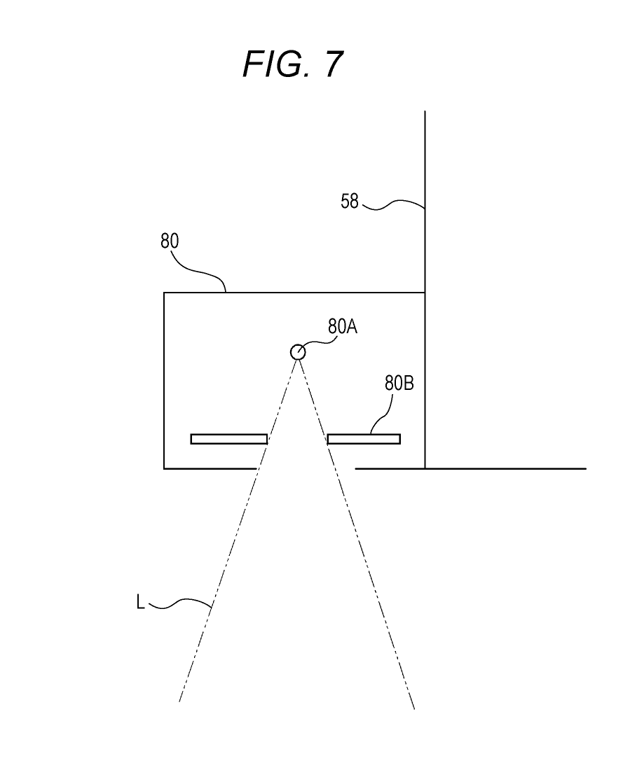

It is possible to configure the light projection apparatus 80 using a lamp, or the like, capable of projecting the visible light L to a predetermined range with substantially uniform intensity, similarly to the irradiation field lamp 65. In this case, it is possible to configure such that, as illustrated in FIG. 7, for example, the light projection apparatus 80 includes an adjustment mechanism 80B having a collimator, or the like, configured to narrow the visible light L projected from a light source 80A and to adjust the above-described range RL (refer to FIG. 5B) indicated by the visible light L.

In this case, in order to enable indication of the range RL (refer to FIG. 5B) including and broader than the irradiation field RX of the radiation X projected from the radiation generator 57 to the subject H, by the visible light L projected from the light projection apparatus 80, the adjustment mechanism 80B is configured to be able to adjust the range RL of the visible light L to be projected, independently from adjustment of the irradiation field RX of the radiation X by the collimator 58A.

It is also possible to configure the light projection apparatus 80 using a laser projection apparatus capable of projecting laser light having a wavelength region of the visible light, as the visible light L. In this case, the laser projection apparatus as the light projection apparatus 80 includes an adjustment mechanism (not illustrated) configured with a computer, a dedicated control section, or the like, to adjust, for example, a target range of the laser light as the visible light L.

Also in this case, in order to enable indication of the range RL including and broader than the irradiation field RX of the radiation X projected from the radiation generator 57 to the subject H, by using the laser light as the visible light L projected from the laser projection apparatus as the light projection apparatus 80, the adjustment mechanism is configured to be able to adjust the range RL of laser light as the visible light L to be projected, independent of adjustment of the irradiation field RX of the radiation X by the collimator 58A.

Alternatively, the light projection apparatus 80 can also be configured with a projection apparatus other than the above-described lamp or the laser projection apparatus.

[Setting of Range Indicated by Visible Light]

Meanwhile, in an embodiment of the present invention, projecting the visible light L from the light projection apparatus 80 during projection of the radiation X, as described above, corresponds to indicating a no-entry range to the nearby person.

Therefore, in consideration of security of the nearby person, it is configured such that the visible light L does not indicate the irradiation field RX itself of the radiation X but indicate the range RL including and broader than the irradiation field RX of the radiation X.

More specifically, it is configured such that the range RL indicated by the visible light L has a broader range than the irradiation field RX of the radiation X in order to provide a margin in the no-entry range so as to avoid a nearby person from being exposed to the radiation X even when a portion of the body of the nearby person enters the range RL to a slight degree.

[Setting Method 1]

In another case where, for example, the light projection apparatus 80 has no adjustment unit such as a collimator for expanding/reducing the range RL of the visible light L, the range RL indicated by the visible light L can be set to a range including and broader than the irradiation field RX applied to a case where the collimator 58A is expanded up to a maximum position and thus the irradiation field RX of the radiation X is expanded up to the maximum range.

With this configuration, not merely in a case where the irradiation field RX of the radiation X is narrowed but also in a case where the irradiation field RX of the radiation X is expanded to its maximum range, the above-described range RL is a range including and broader than the irradiation field RX of the radiation X. Accordingly, by setting the range RL as described above, it is possible to set such that the range RL indicated by the visible light Lisa range including and broader than the irradiation field RX of the radiation X regardless of how the irradiation field RX of the radiation X is set.

[Setting Method 2]

In a case where the light projection apparatus 80 includes the adjustment mechanism 80B having a collimator, or the like, as illustrated in FIG. 7, for example, the range RL indicated by the visible light L can be adjusted by adjusting the opening level of the collimator of the adjustment mechanism 80B, for example. In this case, it is also possible to configure such that the photographer A such as a radiologist manually operates an adjustment unit so as to adjust and set such that the range RL indicated by the visible light L is a range including and broader than the irradiation field RX of the radiation X.

[Setting Method 3]

Moreover, some of the radiation generators 57 have an automatic collimation function (that is, a function to automatically narrow the collimator 58A along operation of the radiation generator 57 to move away from the subject H and the FPD cassette 1). Accordingly, in a case where the radiation generator 57 includes the automatic collimation function, it is also possible to automatically adjust and set the range RL indicated by the visible light L such that the range RL is a range including and broader than the irradiation field RX of the radiation X, by transmitting information related to the narrowing level of the collimator 58A from the radiation generator 57 to the light projection apparatus 80, and performing collimator opening level adjustment of the adjustment mechanism 80B on the light projection apparatus 80 in accordance with the information, for example.

In this case, it is also possible to configure such that the above-described information is transmitted from the radiation generator 57 directly to the light projection apparatus 80, or that the information is transmitted via the console 70 (refer to FIGS. 3 to 5A) .

Other than the above-described setting methods 1 to 3, it is also possible to configure such that the light projection apparatus 80 itself automatically adjusts and sets the range RL indicated by the visible light L by measuring a distance from the light projection apparatus 80 to the FPD cassette 1 (refer to FIG. 5A) by the light projection apparatus 80 itself. In this manner, it is possible to employ appropriate methods for setting the range RL indicated by the visible light L.

[Indication of Range Using Visible Light]

### [Indication of Range Part 1]

When the above-described range RL (refer to FIG. 5B) is indicated by projection of the visible light L from the light projection apparatus 80, it is possible to configure such that the visible light L is projected to a whole region of the above-described range RL. In other words, in this case, similarly to the case where the light L* is projected from the irradiation field lamp 65 to the subject H before photographing, it is possible to configure such that the visible light L is projected to individual portions within the above-described range RL with substantially uniform intensity.

Meanwhile, in this case, when projection of the visible light L is performed, during photographing, as described above, from the light projection apparatus 80 to the range RL, projecting the visible light L in a same manner as in the case of performing projection, before photographing, of the light L* from the irradiation field lamp 65 for positioning, or the like, might lead to confusion, that is, the patient as the subject H, and a nearby person might misunderstand that positioning, or the like, before photographing is currently performed.

Specifically, in a typical case where the light L* is projected from the irradiation field lamp 65 before photographing, the light L* having white color, for example, is projected to individual portions within the irradiation field RX with substantially uniform intensity. In such a case, when it is configured such that the visible light L having the same color as the above-described color (white color, or the like) is projected during photographing from the light projection apparatus 80 to individual portions within the above-described range RL with substantially uniform intensity, the patient as the subject H, and the nearby person, might misunderstand that the photographer A such as a radiologist is performing pre-photographing positioning by projecting the light L* again, believing that the radiation X has not yet been projected. This might lead to a behavior of the patient to move the body, and/or a behavior of the nearby person to allow a portion of the body to enter the range RL.

To avoid this, when the visible light L is projected, as described above, from the light projection apparatus 80 during photographing, it is preferable to configure such that the visible light L is projected from the light projection apparatus 80 in a manner of projection that differs from the manner of projection of the light L* when the light L* is projected from the irradiation field lamp 65 before photographing.

More specifically, at the time of projection of the visible light L from the light projection apparatus 80 during photographing, it is possible to configure such that the visible light L is projected from the light projection apparatus 80 in a manner of projection different from the manner of projection of the light L*, using a method such as reducing the light amount of the visible light L to project, to a level significantly lower than the light amount of the light L* projected from the irradiation field lamp 65 (that is, using the different light amount that enables easy recognition of the visible light L as different from the light L* projected from the irradiation field lamp 65), allowing the visible light L to project to flicker, or varying the color of the visible light L to project.

With this configuration, it is possible to allow the patient as the subject H, and the nearby person, to properly recognize that the photographing is being performed with the radiation X being projected, without causing the above-described confusion or misunderstanding, and thus, it is possible to properly prevent the patient from moving the body, or the nearby person from allowing a portion of the body to enter the range RL.

[Indication of Range 2]

It is also possible to configure to indicate the above-described range RL by projecting the visible light L exclusively to a frame line FL (refer to FIG. 5B) representing a boundary between the above-described range RL and its outside region, from the light projection apparatus 80, instead of projecting the visible light L from the light projection apparatus 80 to the whole region of the above-described range RL, as described above.

With this configuration, while the light L* projected from the irradiation field lamp 65 before photographing is typically projected to the whole region within the irradiation field RX, the visible light L projected from the light projection apparatus 80 is projected exclusively to a portion of the frame line FL. Therefore, when the visible light L is projected from the light projection apparatus 80, it is possible to project the visible light L in a manner of projection that differs from the manner of projection of the light L* when the light L* is projected from the irradiation field lamp 65 before photographing.

Accordingly, it is possible to allow the patient as the subject H, and the nearby person, to properly recognize that the photographing is being performed with the radiation X being projected, without causing the above-described confusion or misunderstanding, and thus, possible to properly prevent the patient from moving the body, or the nearby person from allowing a portion of the body to enter the range RL.

Additionally, when the light projection apparatus 80 is configured with a lamp, or the like, capable of projecting the visible light L as described above, it is possible to achieve the above-described [Indication of range Part 1] by projecting the visible light L to the whole region of the above-described range RL. In contrast, when the light projection apparatus 80 is configured to project the visible light L in a state of the rectangular frame line FL by partially shielding the lamp, or the like, it is possible to achieve the above-described [Indication of range Part 2].

Furthermore, in a case where the light projection apparatus 80 is configured with a laser projection apparatus, as described above, it is possible to achieve the above-described [Indication of range Part 1] by projecting the laser light so as to perform laser light scanning. Still further, by projecting the laser light projected from the laser projection apparatus in such a manner as to move along the above-described frame line FL (such as single-stroke drawing) in a high speed, it is possible to indicate the above-described frame line FL with the laser light, and thus, to achieve the above-described [Indication of range Part 2].

In the description of the present embodiment, the above-described range RL and frame line FL are rectangular as illustrated in FIG. 5B. The present invention, however, is not limited to this, and allows other shapes such as circular and elliptical shapes to be used instead.

[Effects]

According to the radiography system 100 in the present embodiment as described above, it is configured such that the light projection apparatus 80 capable of projecting the visible light L is mounted on the round-visit wagon 50 (or attaching the light projection apparatus 80 to the portable radiation generator 90 (refer to FIG. 10)), the visible light L is projected from the light projection apparatus 80 during photographing, that is during projection of the radiation X from the radiation generator 57 to the subject H, so as to indicate, by the visible light L, the range RL including and broader than the irradiation field RX of the radiation X projected from the radiation generator 57 to the subject H.

Accordingly, the patient as the subject H, and a person other than the patient, who comes in and goes out of the patient room R1, can see the visible light L projected from the light projection apparatus 80, and thus, can properly recognize that the radiation X is projected by checking that the visible light L is projected from the light projection apparatus 80, and recognize that the radiation X is finished by checking that the visible light L is not projected.

Therefore, it is possible to properly avoid problems such that a person other than the patient as the subject H has difficulty in discerning whether projection of the radiation X is finished or be exposed by entering the irradiation field RX of the radiation X or by allowing a portion of the body to enter the irradiation field RX of the radiation X due to misunderstanding that projection of the radiation X is finished.

Furthermore, with the radiography system 100 according to the present embodiment, the visible light L projected from the light projection apparatus 80 is projected, as described above, so as to indicate the range RL including and broader than the irradiation field RX of the radiation X projected from the radiation generator 57 to the subject H.

With this configuration, the person other than the patient as the subject H can properly recognize what range is a no-entry range, that is, recognize the range RL including the irradiation field RX, namely, the projection range of the radiation X, making it possible to prevent the person other than the patient from entering the above-described range RL. Accordingly, it is possible to properly prevent situations such as the person other than the patient as the subject H feels too nervous from not knowing in what range the radiation X is projected and has a false recognition of being prohibited from a range that is actually a permitted range, and thus, feels own action is restricted than necessary so as to avoid entering the area.

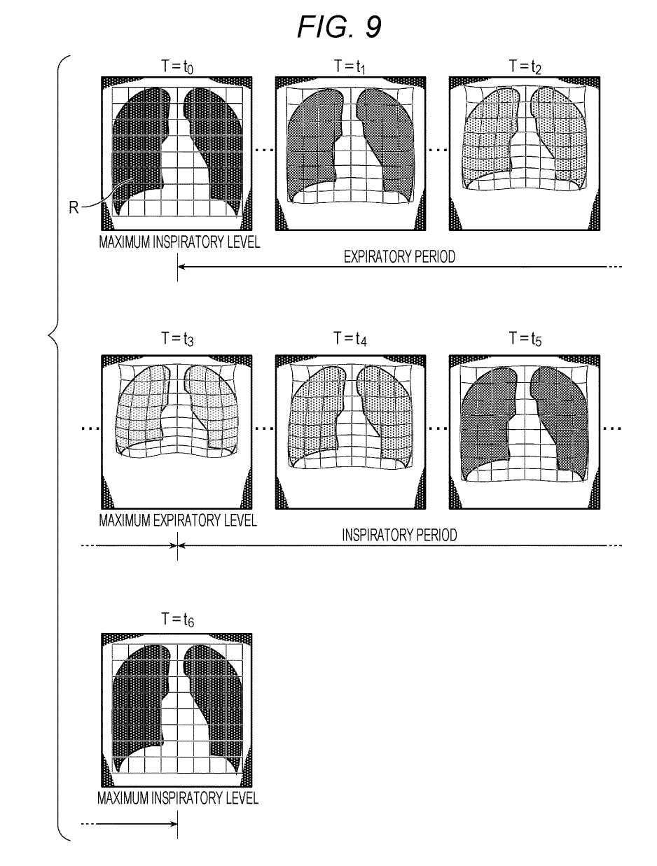

Furthermore, as described above, the photographer A such as a radiologist needs to pay attention by continuously monitoring whether the posture of the patient as the subject H is appropriately maintained during photographing at the time of performing moving image photographing such as kinetic photographing (refer to FIG. 9). With the radiography system 100 according to the present embodiment, the patient as the subject H and the person other than the patient can recognize whether the radiation X is projected, and recognize the range RL including and broader than the irradiation field RX of the radiation X, making it possible to prevent the person other than the patient as the subject H from entering the range RL.

As a result, the photographer A need not be too nervous about the patient as the subject H entering into the irradiation field of radiation, making it possible for the photographer A to concentrate on the patient as the subject H during photographing. In this manner, since there is no more need to pay too much attention to the person other than the patient as the subject H, it is possible to reduce the burden on the photographer A and to properly perform photographing.

[Method of Starting Projection of Visible Light From Light Projection Apparatus]

As a method of starting projection of the visible light L from the light projection apparatus 80 during photographing, it is possible to configure such that the lighting (including blinking; the same will be applied to the following description) is manually started by operation of a switch (not illustrated) on the light projection apparatus 80 by the photographer A such as a radiologist.

In addition, at the time of starting projection of the radiation X from the radiation generator 57, it is possible to configure such that a signal is transmitted from the control section or the console 70, of the radiation generator 57, to the light projection apparatus 80, and that the light projection apparatus 80 is automatically turned on upon receiving this signal.

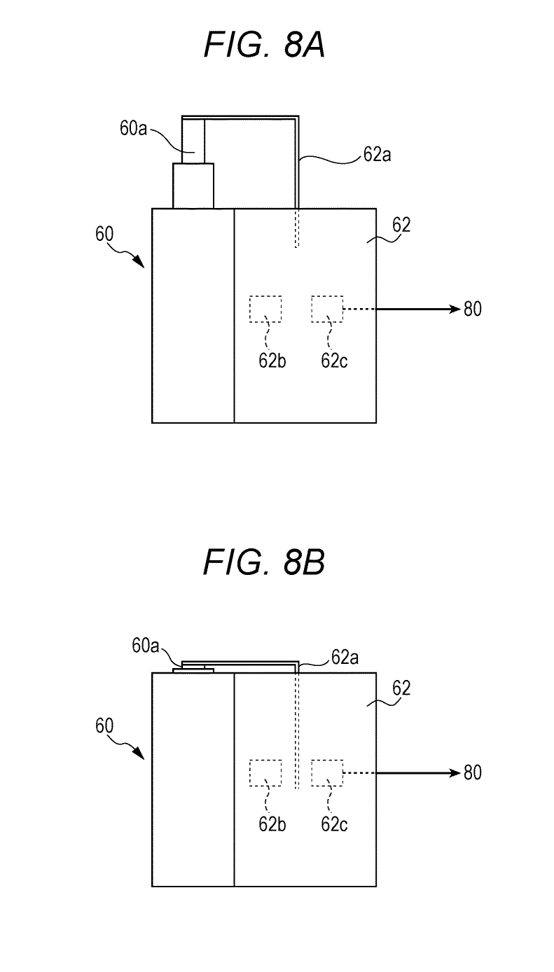

Furthermore, as illustrated in FIGS. 8A and 8B, it is also possible to configure such that a detection apparatus 62 configured to detect pressing of a button 60a of the exposure switch 60 by the photographer A is attached to the exposure switch 60 of the radiation generator 57, and a signal is transmitted to the light projection apparatus 80 (or the signal is transmitted to the light projection apparatus 80 via the console 70, or the like) at a point when the button 60a of the exposure switch 60 is pressed.

In this case, the detection apparatus 62 can be configured, as illustrated in FIGS. 8A and 8B, such that a portion of a movable piece 62a having a substantially L-shape is attached to a tip end of the button 60a of the exposure switch 60. In this case, it is possible to configure such that the movable piece 62a moves when the button 60a of the exposure switch 60 is pressed (fully pressed), and a signal is transmitted to the light projection apparatus 80 when the other end of the movable piece 62a shields projection of light from a light projection element 62b to a light receiving element 62c. Note that the detection apparatus 62 is not limited to this configuration.

It is naturally understandable that the present invention is not limited to the above-described embodiments and modification examples and can be modified as appropriate within the spirit and scope of the present invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. A radiation generator capable of projecting radiation, the radiation generator comprising
a light projection apparatus configured to project visible light that indicates, during projection of radiation from the radiation generator, a range including and broader than an irradiation field of radiation projected from the radiation generator.

2. The radiation generator according to claim 1 further comprising
a collimator configured to narrow the irradiation field of the radiation, wherein
the light projection apparatus is arranged at a position where the visible light projected from the light projection apparatus is not transmitted through an aperture of the collimator.

3. The radiation generator according to claim 1, wherein
the light projection apparatus is configured as an apparatus separate from an irradiation field lamp configured to project, before photographing, light to a region to be an irradiation field of radiation when the radiation is projected to a subject.

4. The radiation generator according to claim 3, wherein,
when the visible light is projected from the light projection apparatus, the visible light is projected from the light projection apparatus, during projection of radiation from the radiation generator, in a manner of projection different from the manner of projection of the light when the light is projected from the irradiation field lamp before projection of radiation from the radiation generator.

5. The radiation generator according to claim 1, wherein
the visible light is projected from the light projection apparatus to a whole region of the range.

6. The radiation generator according to claim 1, wherein
the visible light is projected from the light projection apparatus exclusively to a frame line portion indicating a boundary between the range and a region outside of the range.

7. The radiation generator according to claim 1, wherein
the light projection apparatus is a laser projection apparatus capable of projecting, as the visible light, laser light having a wavelength region of the visible light.

8. The radiation generator according to claim 1, wherein
the light projection apparatus includes an adjustment mechanism capable of adjusting the range of the visible light to project, independently from the irradiation field of the radiation.

9. The radiation generator according to claim 1, wherein
the radiation generator is a variable radiation generator.

10. A round-visit wagon for radiography comprising
a radiation generator capable of projecting radiation, wherein
the radiation generator includes a light projection apparatus configured to project visible light that indicates, during projection of radiation from the radiation generator, a range including and broader than an irradiation field of the radiation projected from the radiation generator.

11. A radiography system comprising:
an FPD cassette including a plurality of radiation detecting elements arranged two-dimensionally; and
a radiation generator capable of projecting radiation, wherein
the radiation generator includes a light projection apparatus configured to project visible light that indicates, during projection of radiation from the radiation generator, a range including and broader than an irradiation field of the radiation projected from the radiation generator.
